(19) United States
(12) Patent Application Publication
(10) Pub. No.: US 2025/0383358 A1
(43) Pub. Date: Dec. 18, 2025

(54) ENZYME-IMMOBLIZED PARTICLES FOR ONLINE PROTEIN DIGESTION

(71) Applicant: Waters Technologies Corporation, Milford, MA (US)

(72) Inventors: Yeliz Tunc Sarisozen, Westford, MA (US); Kevin Wyndham, Worcester, MA (US); Beatrice Muriithi, Attleboro, MA (US); Martin Gilar, Franklin, MA (US); Sabah Pasha, Macclesfield (GB)

(73) Assignee: Waters Technologies Corporation, Milford, MA (US)

(21) Appl. No.: 19/240,955

(22) Filed: Jun. 17, 2025

Related U.S. Application Data
(60) Provisional application No. 63/661,296, filed on Jun. 18, 2024.
(60) Provisional application No. 63/714,386, filed on Oct. 31, 2024.

(51) Int. Cl. G01N 33/68; C12N 9/76; C12N 11/06; C12N 11/084

(52) U.S. Cl. CPC G01N 33/6818; C12N 9/6427; C12N 11/06; C12N 11/084; C12Y 304/21004; G01N 33/6848; G01N 2333/80; G01N 2333/976

(57) ABSTRACT

The present disclosure is directed to nonporous polymer particles having an average particle size of 1 to 10 microns and being functionalized with an enzyme, such as trypsin. The enzyme-immobilized particles, and immobilized enzyme reactors thereof, can be used in methods for on-line protein digestion.

### CROSS REFERENCE TO RELATED APPLICATIONS

The application claims priority to and the benefit of U.S. Provisional Application Ser. No. 63/661,296, filed on Jun. 18, 2024, and U.S. Provisional Application Ser. No. 63/714,386 filed Oct. 31, 2024, both entitled “enzyme-immobilized particles for on-line protein digestion.” The contents of each application are hereby incorporated by reference in their entireties.

### FIELD OF TECHNOLOGY

The present disclosure relates generally to particles conjugated to enzymes, immobilized enzyme reactors comprising said particles, and methods of use thereof.

### BACKGROUND

The characterization of proteins is integral across life sciences industries, from initial research and development to the manufacturing and quality control of protein-based therapeutics (e.g., antibodies and antibody-drug conjugates). Protein characterization typically involves one or more enzymatic treatment steps (i.e., enzymatic proteolysis), affording peptides suitable for analytical methods such as mass spectrometry. Enzymatic proteolysis can be achieved using different approaches, including the use of immobilized-enzyme reactors (IMERs).

IMERs are flow-through devices comprising enzymes immobilized on a solid support. As flow-through devices, IMERs can be integrated with analytical instruments, such as liquid chromatography—mass spectrometry (LC-MS) instruments, to improve workflow efficiency, reduce sample manipulation, and increase sample detection. The composition of the IMER solid support affects residence time and flow—impacting the overall efficacy of the enzymatic digestion and suitability for use in conjunction with downstream analyses.

### SUMMARY OF TECHNOLOGY

The present technology is directed to enzyme-immobilized particles and immobilized enzyme reactors (IMERs) comprising the same for use in on-line protein digestion, i.e., in conjunction with analytical instrumentation including, but not limited to, liquid chromatography—mass spectrometry (LC-MS). The enzyme-immobilized particles of the present technology are nonporous with an average particle size between 1.5 μm to 10 μm and a hydrophilic surface to which one or more enzymes are conjugated. The nonporous morphology and small particle size provide fast mass transfer kinetics and can withstand high pressures (up to, for example, 7000 psi), with low non-specific binding. Thus, IMERs of the present technology can be used for robust and efficient on-line protein digestion.

In one aspect, the present technology is directed to a particle having an average particle size between 1.5 μm to 10 μm and comprising a nonporous polymer core having a hydrophilic surface on an outer layer of the nonporous polymer core to which one or more of an enzyme are conjugated. In some embodiments, the enzyme is trypsin, Lys-C, PNGase F, Asp-N, pepsin, Glu-C, or mixtures thereof. In some embodiments, the nonporous polymer core has a gradient composition. In some embodiments, the nonporous polymer core comprises divinylbenzene (80%). In some embodiments, the hydrophilic surface is selected from the group consisting of (3-glycidyloxypropyl) trimethoxysilane, (3-glycidyloxypropyl) triethoxysilane, polyacrylate, glycidol, glycerol triglycidyl ether, butyl diglycidol ether, and poly (methyl acrylate).

In some embodiments, the one or more enzyme is conjugated to the hydrophilic surface of the particle via an epoxy linker or an aldehyde linker. In some embodiments, the epoxy linker has a formula:

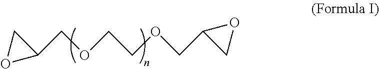

wherein n is an integer from about 1 to about 150. In some embodiments, n is between 1-12. In some embodiments, n is 1. In some embodiments, n is 4. In some embodiments, n is 9.

In some embodiments, the average particle size is between 2 μm to 5 μm. In some embodiments, the average particle size is 3.5 μm. In some embodiments, the enzyme has a surface coverage concentration of between 3 to 9 μg enzyme per mg of particle. In some embodiments, the enzyme is trypsin. In some embodiments, the particle can withstand pressures up to 7000 psi.

In one aspect, disclosed herein is an immobilized enzyme reactor (IMER) comprising a plurality of any one of the particles disclosed herein. In one aspect, disclosed herein is an immobilized enzyme reactor (IMER) comprising a column body formed of a metal or a metal alloy, the column body housing a plurality of any one of the particles disclosed herein. In some embodiments, at least a portion of an interior surface of the column body is coated with a vapor-deposited alkylsilyl material. In some embodiments, the IMER further comprises frits within the column body, wherein the frits are coated with the vapor-deposited alkylsilyl material. In some embodiments, the alkylsilyl material is a hydrophilic, non-ionic layer of polyethylene glycol silane. In some embodiments, the column body comprises a length of about 50 mm and an inner diameter of about 2.1 mm.

In one aspect, disclosed herein is a device comprising an IMER disclosed herein, a column injector positioned upstream of the IMER, and tubing in fluidic connection with and located downstream of the IMER, wherein a portion of an internal surface of the column injector and a portion of an interior surface of the tubing are coated with an alkylsilyl material.

In one aspect, disclosed herein is an on-column method of digesting a sample comprising a protein, the method comprising adding the sample to an IMER of the present technology and incubating the sample, thereby resulting in a digested protein. In some embodiments, incubating the sample is performed at a temperature of between 25° C. to 75° C. In some embodiments, the digested protein is eluted from the IMER.

In some embodiments, the method further comprises one or more pretreatment steps to the sample prior to adding the sample to the IMER. In some embodiments, the one or more pretreatment steps comprises denaturing the protein of the sample, reducing the protein of the sample, alkylating the protein of the sample, and/or desalting the protein of the sample.

In some embodiments, the method results in less than 5% of missed cleavages of the protein of the sample. In some embodiments, the method results in greater than 80% sequence coverage of the protein of the sample. In some embodiments, the method results in greater than 95% sequence coverage of the protein of the sample.

In some embodiments, the method further comprises adjusting the flow rate from an first flow rate to a second flow rate during the incubation step, providing a wait time, then adjusting the flow rate from the second flow rate to a third flow rate. In some embodiments, the first flow rate and the third flow rate are greater than 0 mL/min and the second flow rate is 0 mL/min. In some embodiments, the wait time is from about 0 min to about 3 min.

In some embodiments, the method further comprises submitting the digested protein to downstream analysis comprising liquid chromatography-ultraviolet detection (LC-UV), liquid chromatography-mass spectrometry (LC-MS), or a combination thereof. In some embodiments, the method comprises LC-MS.

### BRIEF DESCRIPTION OF THE DRAWINGS

The technology will be more fully understood from the following detailed description taking in conjunction with the accompanying drawings, in which:

FIGS. 1A-1B provide a cross-sectional illustration of a particle of the present technology. FIG. 1A shows a cross-sectional illustration of a particle prior to attachment of an enzyme. FIG. 1B shows a cross-sectional illustration of a particle after attachment of an enzyme.

FIGS. 2A-2B provide a perspective view of an IMER according to embodiments of the technology. FIG. 2A shows a column body packed with a plurality of particles (i.e., the particles of FIG. 2B), thereby forming an immobilized enzyme reactor (IMER). The cutout section (220) illustrates an interior portion of the IMER. FIG. 2B is a cross-sectional view of the IMER taken along line BB.

FIG. 3 is a schematic illustrating a method of performing on-line protein digestion with an IMER of the present technology.

FIG. 4 is the BAPNA hydrolysis activity performance of trypsin particles.

FIG. 5 is a schematic illustrating the effect of peak parking on analyte diffusion.

FIGS. 6A-6B provide the amino acid sequences for exemplary proteins digested by IMER experiments. Black boxes highlight sequences that were identified by MS. FIG. 6A shows the sequence of human albumin. FIG. 6B shows the sequence for cytochrome C.

### DETAILED DESCRIPTION

In order that the technology may be more readily understood, certain terms are first defined. In addition, it should be noted that whenever a value or range of values of a parameter are recited, it is intended that values and ranges intermediate to the recited values are also part of this disclosure. The word “about” if not otherwise defined means +5%. It is also to be noted that as used herein and in the claims, the singular forms “a,” “and” and “the” include plural references unless the context clearly dictates otherwise.

### Definitions

The term “nonporous” or “nonporous core” as used herein, refers to a material or a material region (e.g., the core) that has a pore volume that is less than 0.1 cc/g. Preferably, nonporous polymer cores have a pore volume that is less than 0.10 cc/g (e.g., 0.05 cc/g), and preferably less than 0.02 cc/g, in some embodiments. Pore volume is determined using methods known in the art based on multipoint nitrogen sorption experiments (Micromeritics ASAP 2400; Micromeritics Instruments Inc., Norcross, GA).

The term “rigid particle,” as used herein, refers to the strength of the particle to withstand applied pressures under flow conditions. A rigid particle appears visually undamaged (i.e., maintains the same form factor without breaking, crushing, or alteration) in a scanning electron microscope image after exposure to pressures of 3,500 psi, wherein less than 10% of the observed particles are visually damaged. In addition, particles in a packed bed that are broken or deformed result in reduced flow and increased pressure as one would predict using the Kozeny-Carmen equation. Broken or deformed particles in a packed bed can increase pressure beyond levels suitable for use in HPLC or UHPLC.

The term “conjugated,” as used herein, refers to the linkage of two molecules formed by the chemical bonding of a reactive functional group of one molecule, such as an enzyme (e.g., trypsin), with an appropriately reactive functional group of another molecule, such as an epoxide.

The term “conjugate,” as used herein, refers to a compound formed by the chemical bonding of a reactive functional group of one molecule, such as enzyme (e.g., trypsin), with an appropriately reactive functional group of another molecule, such as an epoxide. An example of suitably reactive functional groups is a nucleophile/electrophile pair. For instance, the nucleophile may be an amine or thiol group from an amino acid of Protein A, and the electrophile is an epoxide.

The efficiency of enzymatic proteolysis can be determined using downstream analytical methods, including, for example, peptide mapping via mass spectrometry. Common metrics include the percentage of missed cleavages and the percentage of sequence coverage. As used herein, the term “missed cleavage” refers to any uncut bond that has the potential to be cleaved by a proteolytic enzyme. With the use of trypsin as a proteolytic enzyme, a missed cleavage would refer to any uncut lysine/arginine peptide bond. The percentage of missed cleavages refers to the number of missed cleavages, i.e., uncut bonds, relative to the total number of potential cut sites for a respective enzyme. In addition, the extent of sequence coverage of a protein can be determined using peptide mapping methods and the like. As used herein, the term “sequence coverage” refers to the mapping of peptide sequences across the total protein amino acid sequence. As an example, 100% sequence coverage would indicate that peptides were detected that map across 100% of the protein amino acid sequence. 90% sequence coverage would indicate that peptides were detected that map across 90% of the protein amino acid sequence.

Unless defined otherwise, all technical and scientific terms used herein have the same meaning as commonly understood by one of ordinary skill in the art to which this disclosure belongs.

### Particles

To provide stability, surface area, and the appropriate kinetics for enzymatic digestion, the particles of the present technology are nonporous. The nonporous particles provide the appropriate surface area for the attachment or coverage with one or more enzymes. In some embodiments, the particles may be highly spherical and have a smooth surface. In some embodiments, the particles may be highly spherical and have a bumpy convex surface. Such materials have surface areas (measured in m²/g) that are close to their theoretical values. The theoretical surface area for a nonporous smooth sphere is equal to 6/{particle diameter x particle density}. For example, 1 micron polymer particles with a density of approximately 1 g/mL has a theoretical surface area of 6 m²/g, a 3.5 micron polymer particle with the same density has a theoretical surface area of 1.7 m²/g, and a 7 micrometer polymer particle with same density has a theoretical surface area of 0.9 m²/g.

Without wishing to be bound by any particular theory, it is believed that the use of nonporous spheres is advantageous as it improves the kinetics of substrate binding to the enzymes attached to the surface of the sphere (having either a smooth or bumpy with convex surface). It is believed that the form factor of a nonporous sphere shuts down diffusion kinetics into pores of the particles.

The particles of the present technology are nonporous. While some pores or porosity may be incorporated within the particles as discontinuities or as microporosity, nonporous particles are those particles having a pore volume that is less than 0.1 cc/g of the material forming the particle. Preferably, nonporous particles have a pore volume that is less than 0.10 cc/g (e.g., 0.05 cc/g), and preferably less than 0.02 cc/g. in some embodiments. Pore volume is determined using methods known in the art based on multipoint nitrogen sorption experiments (Micromeritics ASAP 2400; Micromeritics Instruments Inc., Norcross, GA).

The particles of the present technology have an average particle size of less than 10 micrometers. For example, the average particle size of a plurality of particles packed within a column in an embodiment of the present technology can be a value anywhere between 8 micrometers and 1.5 micrometers. In one embodiment, the average particle size of the plurality of particles is 7 micrometers. In another embodiment, the average particle size is 3.5 micrometers. In still yet another embodiment, the average particle size is 1.7 micrometers.

The size (i.e., less than 10 micrometers), shape (i.e., spherical), and surface area (i.e., nonporous, smooth or bumpy convex outer surface) create a form factor useful for affinity capture from a flowing sample. To afford high throughput methods and efficient workflows, the particles of the present technology are used in conjunction with LC systems, such as HPLC and UHPLC systems. These systems operate under high pressures (e.g., typically greater than 3,000 psi, such as, for example, 5,000 psi, 7,000 psi, and so forth). As a result, the particles of the present technology need to be rigid particles, such that the particles retain their form factor under HPLC and UHPLC operating conditions.

In general, the particles of the present technology are rigid particles that maintain their form factors (e.g., are not damaged, crushed, squished, or altered) under HPLC or UHPLC operating conditions (e.g., pressures and flow rates). For example, rigid particles in accordance with the present technology, are not visibly altered in form (e.g., not broken, crushed, or altered from spherical) as can be confirmed using scanning electron microscopy before (i.e., control) and after application of HPLC or UHPLC conditions.

A particular material for forming a core (e.g., center or base) of the particles of the present technology that meets the form factor considerations is polymers, and in particular organic polymers. In an embodiment, the nonporous particles of the present technology include a nonporous polymer core. In one embodiment, the nonporous polymer cores of the particles are divinylbenzene (DVB), for example divinylbenzene 80%. In some embodiments, the nonporous polymer cores are formed to include two or more polymers. For example, in some embodiments the nonporous polymer cores include both divinylbenzene and polystyrene. In certain embodiments, the nonporous polymer cores can be manufactured to include a gradient in the polymer composition. For example, the inner portion of the core can be formed of 100% of first polymer (i.e., polymer A) and an outer portion of the core can be formed of 100% or some percentage greater than 0% of a second polymer (i.e., polymer B). Radially from the inner portion to the outer portion of the core, the percentage of polymer A and polymer B can vary to form the gradient in polymer composition. Other embodiments of nonporous polymer cores and particles suitable for use with the present technology are described in U.S. Patent Publication No. 2019/0322783.

While examples and embodiments of the present technology illustrate the use of nonporous polymer cores for the particles, it is noted that other nonporous materials can be utilized as long as the form factor of the particles can be maintained under the operating conditions of HPLC or UPHLC. That is, other materials, such as silica, metal oxides, hybrid inorganic-organic materials, or combinations thereof may be used to create nonporous spherical particles having an average particle size of less than 10 micrometers and that have the rigidity or strength to retain their form factor under the high operating pressures.

To form particles useful for protein digestion, the outer surface of the nonporous core of the particles is linked or connected to an enzyme. To do so, in one embodiment, the outer surface of the nonporous polymer core contains a hydrophilic material. That is, a hydrophilic surface is created on the outer region of the nonporous polymer core. To the hydrophilic surface, one or more molecules of an enzyme is conjugated to the hydrophilic surface. The one or more molecules of an enzyme are able to enzymatically cleave one or more proteins present in a sample. In some embodiments, the enzyme is trypsin, Lys-C, PNGase F, Asp-N, pepsin, or Glu-C. In some embodiments, the particles of the present technology may be conjugated to a heterogenous mixture of trypsin, Lys-C, PNGase F, Asp-N, pepsin, and/or Glu-C.

The hydrophilic surface can also be referred to as a hydrophilic layer. The hydrophilic surface is located on the outer surface of the nonporous polymer core and can be formed of a polymer, molecule or siloxane that has a high density of hydrophilic groups (e.g., hydroxyls, PEG, sugars or carbohydrates). The immobilization of these hydrophilic groups can occur by condensation (ester, amid, silanol, sily ether), polymerization (methacrylates, acrylates, styryl) epoxy activation (epihydrochlorin), or ether formation (direct attachment of PEG or carbohydrate groups by ether formation).

In some embodiments, the hydrophilic surface comprises a material selected from the group consisting of (3-glycidyloxypropyl) trimethoxysilane, (3-glycidyloxypropyl)triethoxysilane, polyacrylate, glycidol, glycerol triglycidyl ether, butyl diglycidol ether, and poly(methyl acrylate)

The one or more molecules of an enzyme can be conjugated to the surface of the particle using a linker. Such linkers include, but are not limited to, epoxy linkers, hydroxyl linkers, and any other linkers as are known in the art (see Hermanson G, “Bioconjugate Techniques” 3^{rd} Edition, July 2013).

In some embodiments, the epoxy linker has a formula:

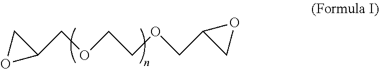

wherein n is an integer from about 1 to about 150. In some embodiments, n is from about 1 to 12. In some embodiments, n is 1, 4, 9, or 12. In some embodiments, n is 1.

In some embodiments, the enzyme has a surface coverage of between 3-9 μg per mg of particle. In some embodiments, the enzyme n has a surface coverage of between 3-3.5 μg per mg of particle, 3.5-4.0 μg per mg of particle, 4.0-4.5 μg per mg of particle, 4.5-5 μg per mg of particle, 5-5.5 μg per mg of particle, 5.5-6 μg per mg of particle, 6-6.5 μg per mg of particle, 6.5-7 μg per mg of particle, 7-7.5 μg per mg of particle, 7.5-8 μg per mg of particle, 8-8.5 μg per mg of particle, or 8.5-9 μg per mg of particle. In some embodiments, the enzyme is trypsin and has a surface coverage of between 3-9 μg per mg of particle. In some embodiments, the enzyme is Lys-C and has a surface coverage of between 3-9 μg per mg of particle. In some embodiments, the enzyme is PNGase F and has a surface coverage of between 3-9 μg per mg of particle. In some embodiments, the enzyme is Asp-N and has a surface coverage of between 3-9 μg per mg of particle. In some embodiments, the enzyme is pepsin and has a surface coverage of between 3-9 μg per mg of particle. In some embodiments, the enzyme is Glu-C and has a surface coverage of between 3-9 μg per mg of particle.

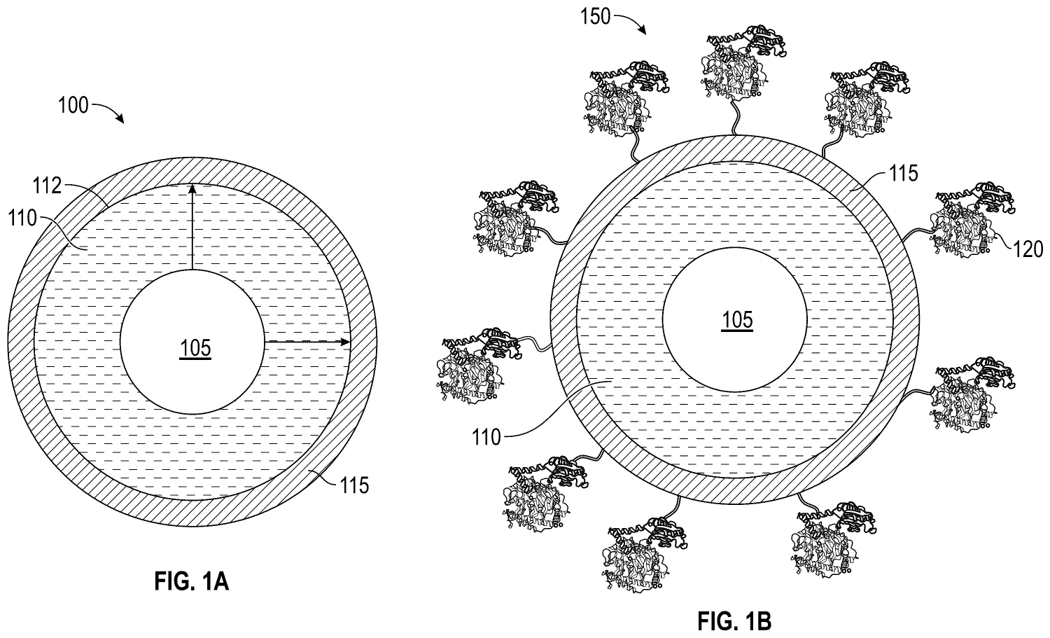

FIG. 1A illustrates an embodiment of a particle having a nonporous core in accordance with the present technology. That is, the particle illustrated in FIG. 1A has a form factor (e.g., spherical, nonporous, and rigid) to withstand operating conditions of HPLC and UHPLC. Particle 100 shown in FIG. 1A is a cross-sectional view prior to the addition of an enzyme, such as, for example, trypsin. Particle 100 includes a nonporous polymer core 112 having an inner core region 105 and a radially extending region 110 surrounding the inner core region 105. The inner core region 105 typically is formed of a polymer or a homogenous blend of polymers, whereas the radially extending region 110 is typically formed of two or more polymers to form a gradient within this region. For example, core region 105 can be formed of polystyrene, whereas radially extending region 110 contains a gradient composition transitioning from 100% polystyrene to 80% to 100% DVB with any remainder being polystyrene.

As illustrated in FIG. 1A, a hydrophilic surface or layer 115 is formed on an outer surface (i.e., opposite to the center region 105) of the nonporous polymer core. In one embodiment, the hydrophilic surface 115 is formed through the application of a hydrophilic primer coating.

Example 1 provides exemplary methods of synthesizing polymer particles that can be functionalized with an enzyme, such as, for example, trypsin, Lys-C, PNGase F, Asp-N, pepsin, and/or Glu-C. Example 1 further describes methods of coating said particles with an epoxy linker for said conjugation, such as an epoxy linker of Formula I. Example 2 describes methods of coating said particles with an aldehyde linker.

To attach the enzyme, e.g., trypsin, a linker is used to conjugate it to the hydrophilic surface 115. FIG. 1B shows a particle 150 after conjugation. That is, particle 150 is the result of conjugating an enzyme 120 to the hydrophilic surface 115 through the use of a linker, such as an epoxy linker of Formula I. One of ordinary skill in the art would understand that there are various ways to attach the enzyme to the hydrophilic layer.

Examples 3 and 4 describe methods of preparing trypsin-conjugated particles using an epoxy linker (Example 3) or an aldehyde linker (Example 4).

Examples 5 and 6 describe a method of preparing trypsin-conjugated particles wherein the trypsin is bound to a reversible inhibitor such as benzamidine. Example 5 describes said methods for trypsin-conjugated particles using an epoxy linker. Example 6 describes said methods for trypsin-conjugated particles using an aldehyde linker.

Example 7 describes an alternative method of preparing trypsin-conjugated particles wherein the trypsin is bound to a reversible inhibitor such as benzamidine. Example 8 characterizes the trypsin surface coverage and the protected surface activity of trypsin in the particles produced by the method of Example 7.

### Immobilized Enzyme Reactors

In one aspect, disclosed herein are immobilized enzyme reactors (IMER) comprising a plurality of the particles disclosed herein. As used herein, the term “immobilized enzyme reactor” refers to a flow-through device that comprises localized enzymes which retain their catalytic activity. As a flow-through device, the immobilized enzyme reactors disclosed herein may be used in conjunction with analytical devices such as liquid chromatography (LC) systems, including high performance LC (HPLC) and ultra-high performance LC (UHPLC) systems, which can further be connected in fluidic to series to one or more detectors, such as a mass spectrometry detector or a fluorescence detector.

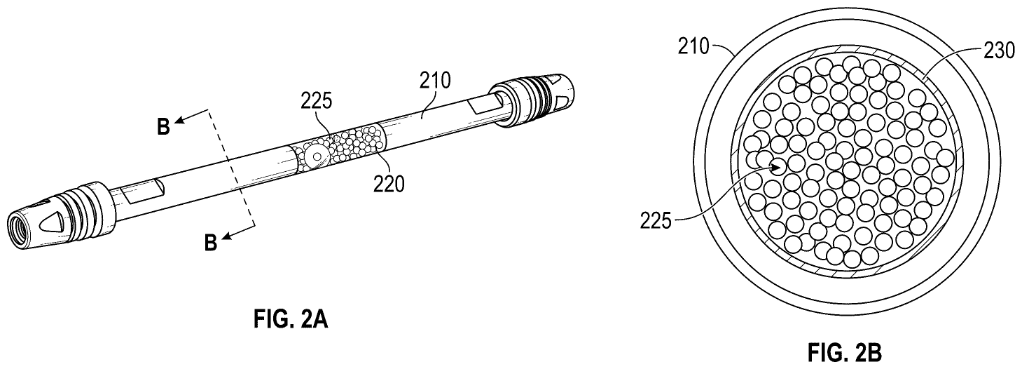

The particles of the present technology can be packed into a number of suitable housings to afford an IMER. In a preferred embodiment, the particles of the present technology are packed into a column body as shown in FIG. 2A. The column body may be formed of a metal or a metal alloy, e.g., titanium or stainless steel. Referring to FIG. 2A, the IMER having a stainless steel column body 210 is packed with a plurality of particles 225. A portion 220 of the column body is removed in FIG. 2A to illustrate the location of a plurality of particles 225. FIG. 2B provides a cross-sectional view of the column body (i.e., the IMER) taken along the line B-B in FIG. 2A.

The cross-sectional view of FIG. 2B illustrates the position of the column body 210 surrounding and housing the plurality of particles 225. In some embodiments, an alkylsilyl coating or other high performance surface is provided to limit or reduce non-specific binding of a sample with the walls or interior surfaces 230 of the column body 210. Without wishing to be bound by theory, it is believed that an alkylsilyl coating covering metal surfaces prevents or minimizes contact between fluids passing through the column body 210 and the interior surfaces 230. The alkylsilyl coating can be applied to the interior surfaces 230 of the metal column body 210 defining what is known as a wetted path of the column. A metal wetted path includes all surfaces formed from metal that are exposed to fluids during use of the IMER. The metal wetted path includes not only the column body walls but also metal frits disposed within the column. In some embodiments, the alkylsilyl coating is applied not only to the walls of the column body 210 but also to the frits.

In general, the alkylsilyl coating is applied through a vapor deposition technique. Vaporized precursors are charged into a reactor in which the part to be coated is located. These vaporized precursors react on the surfaces of the part to be coated to form a first layer of deposited material. The vapor deposition can be applied in a stepwise function to apply a number of layers of deposited material to the surfaces to grow a thickness of the coating and/or to apply layers of different materials (e.g., alternating between a first and second material) to form the coating.

The alkylsilyl coating may be applied to other portions of a liquid chromatography system to which the IMER is connected. For example, the alkylsilyl coating can be applied to metal components residing upstream and downstream of IMER. Specifically, the alkylsilyl coating can be applied to an injector of the liquid chromatography system and to post IMER tubing and connectors (e.g., tubing and connectors leading from the column to downstream components such as detectors). Further, the IMERs of the present technology do not require the addition of additional organic modifiers to reduce non-specific binding. Typically, the addition of an organic modifier (e.g., acetonitrile) may be necessary with IMERs to reduce non-specific binding. Due to the already low non-specific binding of the columns of the present technology, no organic modifier is necessary.

In some embodiments, the alkylsilyl coating comprises a hydrophilic, non-ionic layer of polyethylene glycol silane. In another embodiment, the alkylsilyl coating is formed from one or more of the following precursor materials bis (trichlorosilyl) ethane or bis (trimethoxysilyl) ethane. Other embodiments of alkylsilyl coatings suitable for use with the present technology are described in US Patent Publication No. 2019/0086371 (now U.S. Pat. No. 11,709,155) and US Application Publication No. 2022/0118443.

### Methods of On-line Protein Digestion

The IMERs of the present technology can be used to perform on-line protein digestion of a sample comprising a protein. As used herein, the term “on-line protein digestion” refers to the use of an IMER for protein digestion that is in fluidic connection with a liquid chromatography system such as an HPLC or UHPLC. The terms “on-line” and “on-column” are used interchangeably herein unless described otherwise. For example, but not by way of limitation, an IMER of the present technology may be connected in fluidic series to one or more columns present in an HPLC or UHPLC system.

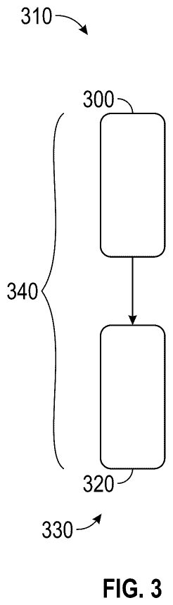

FIG. 3 provides an overview of the method for performing on-line protein digestion of a sample comprising a protein using an IMER (300) of the present technology. The IMER (300) is connected in fluidic series to a chromatography column (320). The chromatography column may be any chromatography column suitable for analysis of peptides, including but not limited to reversed phase chromatography or size-exclusion chromatography. Both the IMER (300) and the chromatography column (320) are connected in fluidic series to a liquid chromatography device (340), such as an HPLC or a UHPLC. A sample (310) comprising a protein is flowed onto the IMER (300). Due to the presence of the enzymes conjugated to the particles of the IMER, the protein in the sample is digested to form peptides. Said peptides are flowed through the IMER (300) to the downstream chromatography column (320). The peptides are then eluted (330) from the chromatography column (320) and can be detected using a suitable detector, such as a mass spectrometry, ultraviolet, or fluorescence detector. In some embodiments, chromatography column (320) is absent. That is, the peptides are eluted (330) directly from the IMER (300) and detected using a suitable detector.

In some embodiments, the sample is added to the IMER (300) and the sample is heated, which increases enzymatic activity. In some embodiments, the IMER is heated to between 25° C. to 75° C. In some embodiments, the IMER is heated to between 25-30° C. 30-35° C., 35-40° C., 40-45° C., 45-50° C., 50-55° C., 55-60° C., 60-65° C., 65-70° C., or 70-75° C.

In some embodiments of the methods provided herein, the sample is pretreated prior to adding the sample to the IMER (300). Pretreatment steps include, but are not limited to, denaturing the protein of the sample, reducing the protein of the sample, alkylating the protein of the sample, and/or desalting the protein of the sample. Methods of pretreating samples are well known in the art and would be readily understood by a person of ordinary skill in the art. For example, a protein sample can be denatured by heating the sample at elevated temperatures (e.g., 70° C., 80° C., 90° C., or higher for a period of time). Protein samples can be reduced via the addition of reducing agents such as beta-mercaptoethanol. Protein samples can be alkylated via the addition of an alkylating agent. Protein samples can be desalted via the use of a desalting column or sorbent.

In some embodiments, an on-column method described herein may include one or more flow rates. For example, the on-column method may include a first flow rate, a second flow rate, and a third flow rate. In some embodiments, the sample is provided to the column at a first flow rate, which is modulated to a second flow rate during the on-column method. In some embodiments, the second flow rate may be modulated to a third flow rate during on-column method.

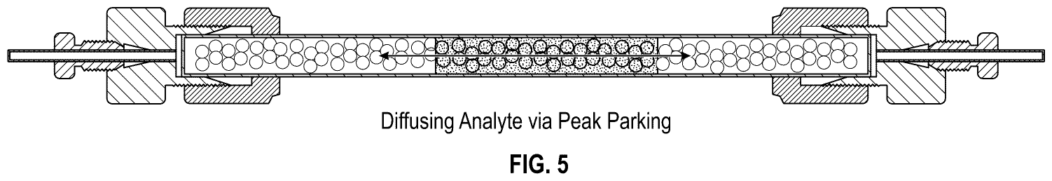

In some embodiments, the method including one or more distinct flow rates includes a first flow rate, a second flow rate, a third flow rate, and a wait time between modulating the first flow rate to the second flow rate and the second flow rate to the third flow rate (alternatively referred to as a “peak parking” method herein). For example, the on-column method may include providing the sample to the chromatography column at a first flow rate (e.g., a flow rate from about 0.1 mL/min to about 1 mL/min; e.g., about 0.1 mL/min, about 0.2 mL/min, about 0.3 mL/min, about 0.4 mL/min, about 0.5 mL/min, about 0.6 mL/min, about 0.7 mL/min, about 0.8 mL/min, about 0.9 mL/min, etc.). After sufficient time for the sample to diffuse through the column, the first flow rate is then modulated to a second flow rate, wherein the second flow rate is less than the first flow rate. Typically, the second flow rate is 0 mL/min. Preferably, the sample has diffused from about 30% to about 70% (e.g., about 50%) of the length of the chromatography column before the first flow rate is modulated to the second flow rate. The second flow rate is then maintained for a wait time. By providing a wait time at a reduced (e.g., 0 mL/min) flow rate, the sample diffuses through the column without the directional force of an applied flow. This in turn causes the concentration of the sample in the vicinity of each enzyme of the IMER to decrease; as well as increasing the time the sample is in contact with the IMER. This results in an improved efficiency of sample digestion by the IMER. FIG. 5 depicts a schematic showing the diffusion of a sample throughout an IMER using a peak parking method. In some embodiments, the wait time is from about 0 min to about 3 min (e.g., about 0.1 min, about 0.2 min, about 0.3 min, about 0.4 min, about 0.5 min, about 0.6 min, about 0.7 min, about 0.8 min, about 0.9 min, about 1 min, about 1.1 min, about 1.2 min, about 1.3 min, about 1.4 min, about 1.5 min, about 1.6 min, about 1.7 min, about 1.8 min, about 1.9 min, about 2 min, about 2.1 min, about 2.2 min, about 2.3 min, about 2.4 min, about 2.5 min, about 2.6 min, about 2.7 min, about 2.8 min, about 2.9 min, etc.). After the wait time, the flow rate may be modulated from the second flow rate to a third flow rate (e.g., a flow rate from about 0.1 mL/min to about 1 mL/min; e.g., about 0.1 mL/min, about 0.2 mL/min, about 0.3 mL/min, about 0.4 mL/min, about 0.5 mL/min, about 0.6 mL/min, about 0.7 mL/min, about 0.8 mL/min, about 0.9 mL/min, etc.). In some embodiments, the third flow rate is equivalent to the first flow rate.

The on-column methods described herein result in robust protein digestion, affording peptides that can be subjected to downstream analyses such as peptide mapping. In some embodiments, the methods provided herein result in a sequence coverage of greater than 80%, greater than 85%, greater than 90%, greater than 95%, or 100% sequence coverage. In some embodiments, the methods provided herein result in less than 10% missed cleavages, less than 5% missed cleavages, less than 1% missed cleavages, or 0% missed cleavages.

### EXAMPLES

### Example 1

Addition of an Epoxy Linker to Hydrophilic, Nonporous, Polymer Particles

Nonporous, epoxy-modified hydrophilic particles were prepared as follows. 1500 g of reagent alcohol (90% ethanol, 5% methanol, and 5% isopropanol), 45.1 g of polyvinylpyrrolidone (PVP-40), 4.8 g of 2,2′-azobiz (2-methylpropionitrile), 5.9 g of Triton™ N-57, and 81.7 g of styrene were charged into a reactor. The reactor was purged with nitrogen gas, heated to 70° C., and stirred for 3 hours. After 3 hours, a solution of 110.4 g of divinylbenzene (DVB) 80%, 39.7 g of PVP-40, 510 g of reagent alcohol, and 100.2 g of p-xylene were added to the reaction mixture at a constant flow rate over two hours. Following this addition, a primer coating solution containing 26.0 g of glycidyl methacrylate (GMA), 26.0 g of ethylene glycol dimethacrylate (EDMA), 36.4 g of PVP-40, and 560 g of reagent alcohol were added to the reaction mixture at a constant flow rate over 1.5 hours. The reaction was maintained at 70° C. for 20 hours, after which the particles were separated from the slurry by filtration. Particles were washed sequentially with methanol, tetrahydrofuran (THF), and acetone. The final product was dried in a vacuum oven at 45° C., resulting in monodisperse 3.5 um polymer particles. Altering concentrations of PVP-40, 2,2′-azobis (2-methylpropionitrile), and Triton™ N-57 can produce particles that range in size.

The resultant. 3.5 um polystyrene/DVB particles with the poly (GMA/EDMA) primer were coated with a hydrophilic layer. 70 g of the particles were hydrolyzed in 0.5 M H₂SO₄ at 60° C. for 1 to 20 hours. The hydrolyzed particles were washed sequentially with water and methanol and dried under vacuum at 45° C. overnight. The dried particles were added to a 1 L three-necked round bottom flask with an overhead stirring motor, stirring shaft, and stir blade, a water-cooled condenser, a nitrogen inlet, and a probe-controlled heating mantle. 700 mL of anhydrous diglyme (diethylene glycol dimethyl ether) was added to the flask, after which the flask was sealed and purged with nitrogen gas for 15 minutes with moderate stirring. 2.0 g of potassium tert-butoxide was added and the reaction raised to 70° C. A mixture of 10.5 g glycidol, 2.6 g glyceroltriglycidyl ether, and 14.9 g of anhydrous diglyme was added to the mixture in four equal aliquots at 30 minute intervals. The reaction was held at 70° C. for 2 hours, cooled to RT, and filtered. The resultant particles were washed sequentially with water six times, methanol 3 times, and dried under vacuum overnight at 45° C. The following procedure results in a hydrophilic layer that is 2-4% by weight of the entire particle.

The epoxy linker can be added as follows. 100 g of ethylene glycol diglycidyl ether (EGDGE) and 100 g of methanol was added to 20 g of the resultant 3.5 um particles at RT. 1 mL of 50% sodium hydroxide in water was added and the reaction was stirred continuously for 20 hours. The particles were isolated by filtration, washed with 40 mL of methanol 10 times, and partially dried under nitrogen flow. Particles were stored for later use in a methanol wet bed at 4° C.

Alternatively, 100 g of poly (ethylene glycol) diglycidyl ether (PEGDE 200; the epoxy linker of Formula I wherein n is 4) and 100 g of methanol was added to 20 g of the resultant 3.5 um particles at RT. 1 mL of 50% sodium hydroxide in water was added and the reaction was stirred continuously for 20 hours. The particles were isolated by filtration, washed with 40 mL of methanol 10 times, and partially dried under nitrogen flow. Particles were stored for later use in a methanol wet bed at 4° C.

Alternatively, 100 g of poly (ethylene glycol) diglycidyl ether (PEGDE 400; the epoxy linker of Formula I wherein n is 9) and 100 g of methanol was added to 20 g of the resultant 3.5 um particles at RT. 1 mL of 50% sodium hydroxide in water was added and the reaction was stirred continuously for 20 hours. The particles were isolated by filtration, washed with 40 mL of methanol 10 times, and partially dried under nitrogen flow. Particles were stored for later use in a methanol wet bed at 4° C.

### Example 2

### Addition of an Aldehyde Linker to Hydrophilic, Nonporous, Polymer Particles

Any of the above particles in Example 1 may further be hydrolyzed with 0.5M acetic acid at 70° C. for 20 hours. The hydrolyzed particles are washed with water until the pH of the supernatant increases above 5. For diol oxidation, water-wet particles are then dispersed in 0.02 M sodium periodate solution (˜1 g particles per 10 mL solution) and stirred at RT for 1 hour. The resultant particles are isolated by filtration. washed with water, and dried under nitrogen flow.

### Example 3

### Preparation of Trypsin-Immobilized Particles Using Epoxide Linker

The particles of Example 1 were functionalized with trypsin using the following methods.

Method with Salting Out Reagent

1.5 g of particles prepared as in Example 1 were mixed in 8.3 mL of 100 mM carbonate-bicarbonate buffer (pH 10). 0.3 mL of a 50 mg/mL solution of trypsin (15 mg) was added to the mixture. 21.4 mL of a buffer containing 1.4 M sodium sulfate (a salting out agent) was added dropwise to the solution and stirred for 20 hours at 37° C. Following the 20-hour incubation, 1 g of ethanolamine in 4 mL of buffer was added and the reaction was stirred at RT for 3 hours. Particles were isolated by filtration and washed sequentially three times with water (pH 4, adjusted with HCl), twice with water (pH 7), and twice with 100 mM PBS (pH 8), 0.02% sodium azide (storage buffer). Particles were stored in the storage buffer (˜10 mL buffer per gram of particle) at 4° C. Trypsin coverage of the particles was determined using a standard bicinchoninic acid assay (BCA) and shown in Table 1, as product 3a.

Method without Salting Out Reagent

1.5 g of particles prepared as in Example 1 were mixed in 29.7 mL of 100 mM carbonate-bicarbonate buffer (pH 10). 0.3 mL of a 50 mg/mL solution of trypsin (15 mg) was added to the solution and stirred for 20 hours at 37° C. Following the 20-hour incubation, 1 g of ethanolamine in 4 mL of buffer was added and the reaction was stirred at RT for 3 hours. Particles were isolated by filtration and washed sequentially three times with water (pH 4, adjusted with HCl), twice with water (pH 7), and twice with 100 mM PBS (pH 8), 0.02% sodium azide (storage buffer). Particles were stored in the storage buffer (˜10 mL buffer per gram of particle) at 4° C. Trypsin coverage of the particles was determined using a standard bicinchoninic acid assay (BCA) and shown in Table 1, as product 3b.

### Example 4

### Preparation of Trypsin-Immobilized Particles Using Aldehyde Linker

The particles of Example 2 were functionalized with trypsin using the following methods.

Method with Salting Out Reagent

1.4 g of particles prepared as in Example 2 were mixed in 28 mL of 100 mM carbonate-bicarbonate buffer with 1.3 M sodium sulfate (pH 10). 0.28 mL of 50 mg/mL solution of trypsin (14 mg) was added to the mixture and incubated with gentle mixing for 3 hours at RT. Following the 3-hour incubation, the suspension was filtered, washed twice with 100 mM PBS (pH 7.2), and suspended in the 100 mM PBS buffer (˜20 mL buffer per gram of particle). The mixture was transferred to a reaction flask. 0.1 g of sodium cyanoborohydride in 0.4 mL of 1 M sodium hydroxide solution was added to the reaction and stirred at RT for 2 hours. After 2 hours, 1 g of ethanolamine in 3.5 mL buffer was added and the reaction stirred for 1 hour. Particles were isolated by filtration, washed sequentially with water four times, and storage buffer twice. Particles were stored in the storage buffer (˜10 mL buffer per gram of particle at 4° C. Trypsin coverage of the particles was determined using a standard bicinchoninic acid assay (BCA) and shown in Table 1, as product 4a.

Method without Salting Out Reagent

1.4 g of particles prepared as in Example 2 were mixed in 28 mL of 100 mM carbonate-bicarbonate buffer (pH 10). 0.28 mL of 50 mg/mL solution of trypsin (14 mg) was added to the mixture and incubated with gentle mixing for 3 hours at RT. Following the 3-hour incubation, the suspension was filtered, washed twice with 100 mM PBS (pH 7.2), and suspended in the 100 mM PBS buffer (˜20 mL buffer per gram of particle). The mixture was transferred to a reaction flask. 0.1 g of sodium cyanoborohydride in 0.4 mL of 1 M sodium hydroxide solution was added to the reaction and stirred at RT for 2 hours. After 2 hours, 1 g of ethanolamine in 3.5 mL buffer was added and the reaction stirred for 1 hour. Particles were isolated by filtration, washed sequentially with water four times, and storage buffer twice. Particles were stored in the storage buffer (˜10 mL buffer per gram of particle) at 4° C. Trypsin coverage of the particles was determined using a standard bicinchoninic acid assay (BCA) and shown in Table 1, as product 4b.

TABLE 1
Particles Functionalized with Streptavidin
with and without salting out

| | | | Trypsin |
|---|---|---|---|
| | | | Coverage |
| Product | | | (μg/mg |
| # | Linker | Salting Out | particle) |
| 3a | Epoxide | Yes | 4.0 |
| 3b | Epoxide | No | 1.6 |
| 4a | Aldehyde | Yes | 3.8 |
| 4b | Aldehyde | No | 2.8 |

While the above methods utilize trypsin as the enzyme, it is understood that the above methods can be used to functionalize particles of the present technology with other enzymes, including Lys-C, PNGase F, Asp-N, pepsin, and/or Glu-C.

### Example 5

### Preparation of Trypsin-Immobilized Particles with Epoxide Linker in Presence of a Reversible Inhibitor

2 g of particles prepared as in Example 1 were mixed in 10.5 mL of 100 mM carbonate-bicarbonate buffer (pH 10). 1 to 2.5 mL of a 40 mg/mL solution of trypsin and 0.1 to 0.4 mL of 200 mM benzamidine hydrochloride (a reversible inhibitor of trypsin) was added to the mixture. 28.5 mL of 100 mM carbonate-bicarbonate buffer (pH 10) containing 1.5 M sodium sulfate (a salting out agent) was added dropwise to the solution and stirred for 5 hours at 25 to 37° C. Following the 5-hour incubation, 1.4 g of ethanolamine in 5mL of buffer was added and the reaction stirred at RT for 1 hour. Particles were isolated by filtration and washed sequentially three times with 0.5 M NaCl solution at pH 4 (adjusted with HCl), three times with water, and twice with 50 mM Tris-HCl buffer with 10 mM CaC12 and 0.02% sodium azide, pH7.4 (storage buffer). Particles were stored in the storage buffer (˜10 mL buffer per gram of particle) at 4° C. Trypsin coverage of the particles was determined using a standard bicinchoninic acid assay (BCA). The final concentration of trypsin, benzamidine hydrochloride, and the temperature of the reaction can be adjusted to manipulate the extent of streptavidin coverage on a given particle, as shown in Table 2.

While the above methods utilize trypsin as the enzyme, it is understood that the above methods can be used to functionalize particles of the present technology with other enzymes, including Lys-C, PNGase F, Asp-N, pepsin, and/or Glu-C.

### Example 6

### Preparation of Trypsin-Immobilized Particles with Aldehyde Linker in Presence of Inhibitor

The particles of Example 2 can be functionalized to include Trypsin in the presence of an inhibitor.

2 g of particles prepared as in Example 2 were mixed in 40 mL of 100 mM carbonate-bicarbonate buffer containing 1.5 M sodium sulfate (pH 10). 0.4 to 0.8 mL of a 50 mg/mL solution of trypsin and 0.1 to 0.4 mL of 200 mM benzamidine hydrochloride was added to the mixture and stirred for 3 hours at 23° C. Following the 3 h incubation, the suspension was filtered and washed with 100 mM PBS, pH7.2 twice and suspended in the same buffer (1 g particles per 20 mL buffer) and transferred to a reaction flask. Next, 0.15 g sodium cyanoborohydride in 0.6 mL of 1 M sodium hydroxide solution was added and the reaction was stirred at RT for 2 hours. Following 2 hours reaction, 1.4 g ethanolamine in 5 mL buffer was added and the reaction was stirred at RT for 1 hour. Particles were isolated by filtration and washed sequentially three times with 0.5 M NaCl solution at pH 4 (adjusted with HCl), twice with water, and twice with 50 mM Tris-HCl buffer with 10 mM CaCl2 and 0.02% sodium azide, pH7.4 (storage buffer). Particles were stored in the storage buffer (˜10 mL buffer per gram of particle) at 4° C. Trypsin coverage of the particles was determined using a standard bicinchoninic acid assay (BCA). The final concentration of trypsin and benzamidine hydrochloride of the reaction can be adjusted to manipulate the extent of streptavidin coverage on a given particle, as shown in Table 2.

TABLE 2
Particles Functionalized with Streptavidin in presence of inhibitor

| | | Trypsin | Benzamidine | | Trypsin |
|---|---|---|---|---|---|
| | | Concen- | Concen- | Reaction | Coverage |
| Product | | tration | tration | Temperature | (μg/mg |
| # | Linker | (mg/mL) | (mM) | (° C.) | particle) |
| 5a | Epoxide | 2.5 | 0.5 | 37 | 13.5 |
| 5b | Epoxide | 1.0 | 2.0 | 37 | 4.2 |
| 5c | Epoxide | 2.5 | 2.0 | 25 | 5.2 |
| 5d | Epoxide | 1.0 | 0.5 | 25 | 2.2 |
| 6a | Aldehyde | 0.5 | 0.5 | 23 | 3.6 |
| 6b | Aldehyde | 1.0 | 2.0 | 23 | 4.3 |
| 6c | Aldehyde | 1.0 | 0.5 | 23 | 4.5 |
| 6d | Aldehyde | 0.5 | 2.0 | 23 | 3.4 |

While the above methods utilize trypsin as the enzyme, it is understood that the above methods can be used to functionalize particles of the present technology with other enzymes, including Lys-C, PNGase F, Asp-N, pepsin, and/or Glu-C.

### Example 7

### Alternative Preparation of Trypsin-Immobilized Particles with Aldehyde Linker in Presence of Inhibitor

An alternative method for functionalizing the particles of Example 2 with Trypsin in the presence of an inhibitor is now described.

3 g of particles prepared as in Example 2 were mixed in 6 mL of 100 mM carbonate-bicarbonate buffer. To this, 1.2 mL of a 50 mg/mL solution of trypsin and 0.6 mL of 200 mM benzamidine hydrochloride were added. Next, 52.8 mL of buffer containing 1.5 M sodium sulfate (pH 10) was added to the mixture and stirred for 2 hours at 23° C. Following the 2 h incubation, the suspension was filtered and washed with 100 mM PBS, pH7.2 twice and suspended in the same buffer (1 g particles per 20 mL buffer) and transferred to a reaction flask. To this, 0.6 mL of 200 mM benzamidine hydrochloride were added. Next, a 0.8 mL of 4.0 M sodium cyanoborohydride in 1 M sodium hydroxide solution was added and the reaction was stirred at RT for 2 hours. Following 2 hours reaction, 2.1 g ethanolamine in 7.3 mL buffer was added and the reaction was stirred at RT for 1 hour. Particles were isolated by filtration and washed sequentially three times with 0.5 M NaCl solution at pH 4 (adjusted with HCl), twice with water, and twice with 50 mM Tris-HCl buffer with 10 mM CaC12 and 0.02% sodium azide, pH7.4 (storage buffer). Particles were stored in the storage buffer (˜10 mL buffer per gram of particle) at 4° C.

### Example 8

### Enzyme Activity Test of Immobilized Trypsin using BAPNA as Substrate

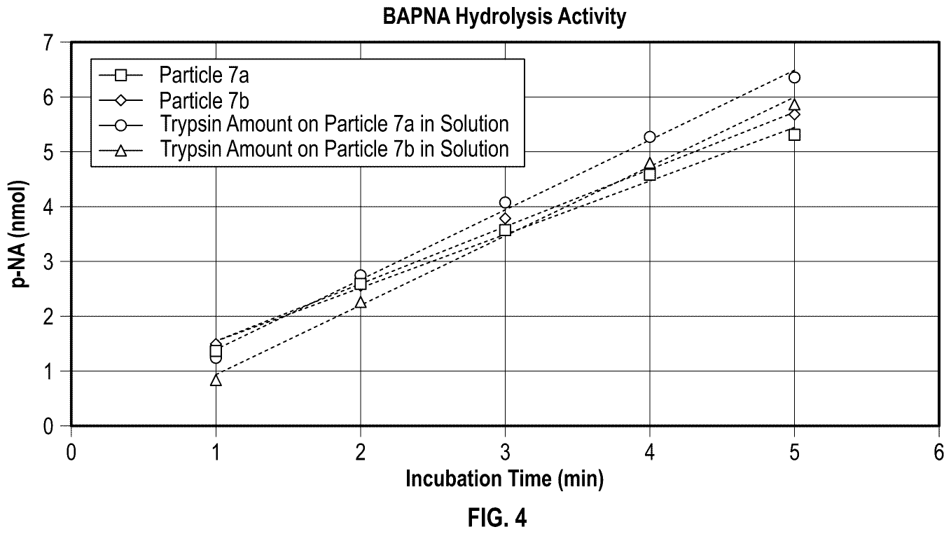

The enzyme activity and surface coverage of trypsin in the particles of Example 7 was then determined. The surface coverage of Trypsin on the particles was determined using a standard bicinchoninic acid assay (BCA), and is reported in Table 3. Enzyme activity was determined using the enzymatic cleavage of Nα-Benzoyl-D, 1-arginine 4-nitroanilide hydrochloride (BAPNA) to p-nitroaniline (p-NA) and benzoyl arginine as follows. Fresh % 0.1 BAPNA aqueous solution and trypsin-immobilized particle suspension in 1 mM HCl solution (around 50 mg/mL) were prepared. 0.3 mL of the suspension was mixed with 2.4 mL 50 mM Tris-HCl buffer with 10 mM CaCl2, pH7.4. Next, BAPNA solution was added to the suspension and mixed by inversion. 0.4 mL sample was taken from the suspension at 1-minute time intervals and filtered right away for the first 5 minutes. 150 μL sample from the filtrate sample was transferred to 96-well plate and the absorbance of the released p-NA was measured at 405 nm and the concentration of p-NA was determined using p-NA standard calibration curve. The results are summarized in FIG. 4. The enzyme activity of the immobilized trypsin was compared to the enzyme activity of free trypsin. The percentage of enzyme activity of the immobilized trypsin is reported as % protected activity, and is reported in Table 3.

TABLE 3
Particles Functionalized with Streptavidin in Presence of Inhibitor

| | | Trypsin | |
|---|---|---|---|
| | | Coverage | |
| Product | | (μg/mg | % Protected |
| # | Linker | particle) | Activity |
| 7a | EGDGE | 5.4 | 82 |
| 7b | PEGDGE 400 | 5.8 | 76 |

### Example 9

### On-Column Digestion of Human Alumin With Trypsin-Immobilized Particles

Trypsin-immobilized particles as prepared in Example 7 were evaluated for the on-column digestion of human Albumin. 200 μL of a 1 mg/mL solution of human albumin in water was prepared. The sample was reduced by addition of 10 μL of 0.2 M dithiothreitol followed by stirring for 1 hr at 750 rpm and 60° C. The sample was then alkylated through the addition of 10 μL of 0.375 M iodoacetamide at room temperature and stirring for 30 minutes.

5 μL of sample was then injected onto a 2.1 mm×50 mm chromatography column including the trypsin-immobilized particles as prepared in Example 7 using a mobile phase of 0.1% formic acid in water at a flow rate of 0.2 mL/min. The sample was then digested using a peak parking method. The peak parking method included maintaining the flow rate at 0.2 mL/min for about 0.24 min, allowing the sample to diffuse partially thorough chromatography column. At 0.25 min, the flow rate was reduced to 0 mL/min. The reduced flow rate was maintained until 1.49 minutes. At 1.5 minutes, the flow rate was increased to 0.2 mL/min, and the sample was eluted from the chromatography column until 3 min.

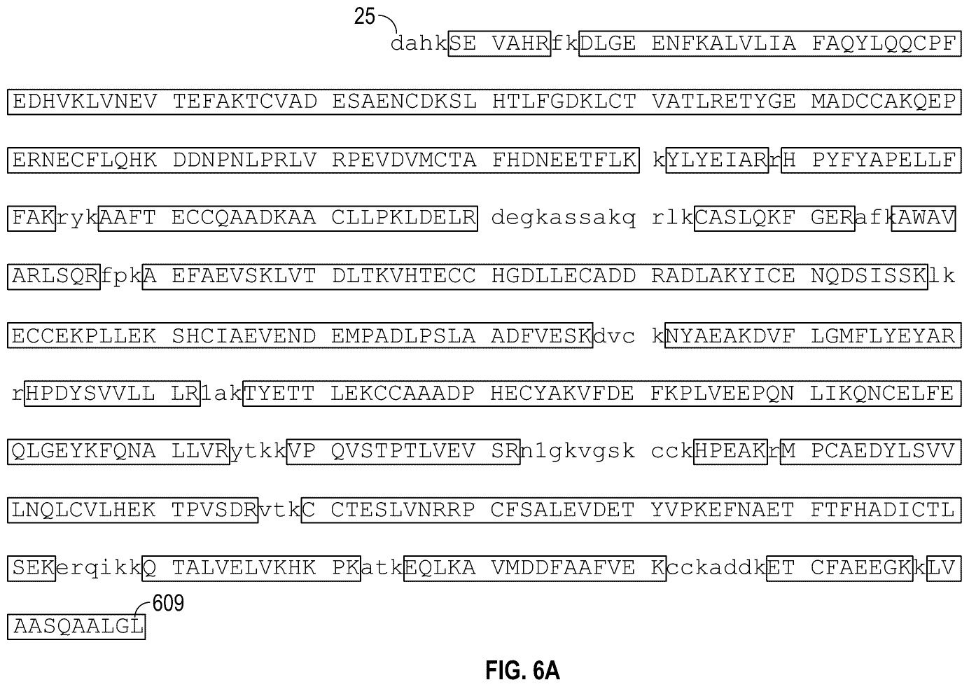

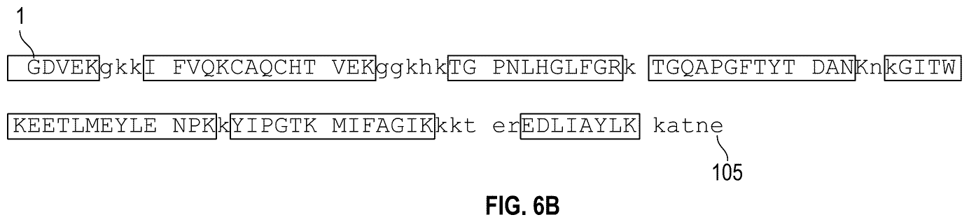

After elution from the chromatography column, the sample was analyzed with a mass spectrometer. The identified sequences are shown in FIG. 6A. FIG. 6A highlights the amino acid sequence ranging from the aspartic acid at position 25 (Asp25; see 25 in FIG. 6A) to the leucine at position 609 (Leu609; see 609 in FIG. 6A). 82% of the maximum sequence coverage of human albumin was detected with high confidence in mass spectrometry. Furthermore, the results were observed with an experiment time of at most 3 minutes, with only 1.25 minutes of peak parking provided.

### Example 10

### On-Column Digestion of Cytochrome C With Trypsin-Immobilized Particles

Trypsin-immobilized particles as prepared in Example 7 were evaluated for the on-column digestion of cytochrome C. 200 μL of a 1 mg/mL solution of equine heart derived cytochrome C was prepared in water.

A 5 μL sample was then injected onto a 2.1 mm×50 mm chromatography column including the trypsin-immobilized particles as prepared in Example 7 using a mobile phase of 0.1% formic acid in water at a flow rate of 0.2 mL/min. The sample was then digested using a peak parking method. The peak parking method included maintaining the flow rate at 0.2 mL/min for about 0.24 min, allowing the sample to diffuse partially thorough chromatography column. At 0.25 min, the flow rate was reduced to 0 mL/min. The reduced flow rate was maintained until 1.49 minutes. At 1.5 minutes, the flow rate was increased to 0.2 mL/min, and the sample was eluted from the chromatography column until 3 min.

After elution from the chromatography column, the sample was analyzed with a mass spectrometer. The amino acid sequence of cytochrome C is shown in FIG. 6B. FIG. 6B highlights the amino acid sequence ranging from the glycine at position 1 (Gly1; see 1 in FIG. 6B) to the glutamic acid at position 105 (Glu105; see 105 in FIG. 6B). 94% of the maximum sequence coverage of cytochrome C was detected by automated proteomic data analysis. Two additional sequences originating from cytochrome C were detected. The first corresponds to an acetylated derivative of the sequence ranging from the glycine at position 1 (Gly1;) to the lysine at position 5 (Lys5) of cytochrome C. The second corresponds to the haem-bound region of cytochrome C corresponding to the cystine at position 14 (Cys14) to the lysine at position 22 (Lys22). Including these derivatives, 99% of the maximum sequence coverage of cytochrome C was observed with high confidence. FIG. 6B highlights the identified amino acids.

The preceding results were obtained with high confidence in less than an experiment time of at most 3 minutes, with only 1.25 minutes of peak parking provided.

1. A particle comprising:
a nonporous polymer core;
a hydrophilic surface on an outer layer of the nonporous polymer core; and
one or more of an enzyme conjugated to the hydrophilic surface,
wherein the particle has an average particle size between 1.5 μm to 10 μm.

2. The particle of claim 1, wherein the enzyme is trypsin, Lys-C, PNGase F, Asp-N, pepsin, Glu-C, or mixtures thereof.

3. The particle of claim 1, wherein the nonporous polymer core has a gradient composition.

4. The particle of claim 1, wherein the nonporous polymer core comprises divinylbenzene (80%).

5. The particle of claim 1, wherein the hydrophilic surface is selected from the group consisting of: (3-glycidyloxypropyl) trimethoxysilane, (3-glycidyloxypropyl) triethoxysilane, polyacrylate, glycidol, glycerol triglycidyl ether, butyl diglycidol ether, and poly (methyl acrylate).

6. The particle of claim 1, wherein the one or more enzyme is conjugated to the hydrophilic surface of the particle via an epoxy linker or an aldehyde linker.

7. The particle of claim 6, wherein the epoxy linker has a formula: 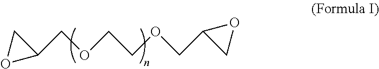
wherein n is an integer from about 1 to about 150.

8. (canceled)

9. (canceled)

10. (canceled)

11. (canceled)

12. The particle of claim 1, wherein the average particle size is between 2 μm to 5 μm.

13. (canceled)

14. The particle of claim 1, wherein the enzyme has a surface coverage concentration of between 3-9 μg enzyme per mg of particle.

15. The particle of claim 14, wherein the enzyme is trypsin.

16. The particle of claim 15, wherein a plurality of the trypsin enzyme are bound to an inhibitor, wherein the inhibitor is benzamidine.

17. (canceled)

18. (canceled)

19. (canceled)

20. An immobilized enzyme reactor (IMER) comprising:
a column body formed of a metal or a metal alloy, the column body housing a plurality of the particles of claim 1.

21. The IMER of claim 20, further comprising frits within the column body, wherein the frits and/or at least a portion of an interior surface of the column body is coated with a vapor-deposited alkylsilyl material.

22. (canceled)

23. The IMER of claim 21, wherein the vapor-deposited alkylsilyl material is a hydrophilic, non-ionic layer of polyethylene glycol silane.

24. (canceled)

25. (canceled)

26. An on-column method of digesting a sample comprising a protein, the method comprising:
adding the sample to the IMER of claim 20; and
incubating the sample, thereby resulting in digested protein.

27. The on-column method of claim 26, wherein incubating the sample is performed at a temperature of between 25° C. to 75° C.

28. (canceled)

29. The on-column method of claim 26, further comprising one or more pretreatment steps to the sample prior to adding the sample to the IMER, the one or more pretreatment steps comprising denaturing the protein of the sample, reducing the protein of the sample, alkylating the protein of the sample, and/or desalting the protein of the sample.

30. (canceled)

31. (canceled)

32. The on-column method of claim 26, wherein the method results in greater than 80% sequence coverage of the protein of the sample.

33. (canceled)

34. The on-column method of claim 26, wherein the method further comprises adjusting the flow rate from a first flow rate to a second flow rate during the incubation step, providing a wait time, then adjusting the flow rate from the second flow rate to a third flow rate.

35. (canceled)

36. The on-column method of claim 26, wherein the method further comprises submitting the digested protein to downstream analysis comprising liquid chromatography-ultraviolet detection (LC-UV), liquid chromatography-mass spectrometry (LC-MS), or a combination thereof.

37. (canceled)
